(19) United States
(12) Patent Application Publication
(10) Pub. No.: US 2006/0058696 A1
(43) Pub. Date: Mar. 16, 2006

(54) Air sampling apparatus with related sensing and analysis mechanism

(75) Inventor: Steven D. Hamilton, Greenfield, WI (US)

Correspondence Address: RYAN KROMHOLZ & MANION, S.C., POST OFFICE BOX 26618, MILWAUKEE, WI, 53226, US

(73) Assignee: QuinTron Instrument Company

(21) Appl. No.: 10/938,859

(22) Filed: Sep. 10, 2004

(51) Int. Cl. A61B 5/08

(52) U.S. Cl. 600/532; 600/543

(57) ABSTRACT

An air sampling apparatus is disclosed, including a bag for containing air and a sensing mechanism coupled to said bag. The self contained sampling and sensing mechanism can be used for visual or other detection of targeted analytes within a breath.

### BACKGROUND OF THE INVENTION

This invention relates to the field of sampling air from the lungs and specifically to the field of obtaining a sample of a person's air, including alveolar air from the alveoli of the lungs of a person.

Air from the lungs of a person can be used for many different types of testing that would otherwise require the person to undergo an invasive procedure. For example, alveolar air can be analyzed for, but not limited to, the noninvasive diagnosis of a wide variety of conditions including the noninvasive diagnosis of stomach infections related to a high incidence of ulcers, enzymatic deficiencies, and metabolic conditions and/or abnormalities. Crucial to any such testing is the ability to get an accurate sample containing a sufficient volume of air representative of true alveolar air, necessary for specific testing.

A simple to use, inexpensive, and user-friendly apparatus is desired to collect and store human breath samples.

### SUMMARY OF THE INVENTION

The present invention incorporates one or more sensing and analysis mechanisms by coupling said mechanisms to a breath collection apparatus, such as a bag. One exemplary bag is described in commonly owned U.S. Pat. No. 6,468,477.

Preferred sensing and analysis mechanisms are vapor-selective detectors or “artificial noses” that are typically based upon the production of an interpretable signal or display upon exposure to a vapor emitting substance or odorant (hereinafter sometimes referred to as an “analyte”) within a humans breath. More specifically, typical artificial noses are based upon selective chemical binding or an interface between a detecting compound of the artificial nose and an analyte or odorant, and then transforming that chemical binding into a signal or display, i.e., signal transduction.

### BRIEF DESCRIPTION OF THE DRAWINGS

FIG. 1 is a side view of a breath collection bag of the present invention, coupled to a sensing mechanism for color analysis.

FIG. 2A is a side view, with portions cut away, of an alternate embodiment of a breath collection bag, coupled to a sensing mechanism for color analysis.

FIG. 2B is a cross sectional view of a breath collection bag of the present invention shown in FIG. 1, coupled to a sensing mechanism for color analysis.

FIG. 3 is a side view of a breath collection bag of the present invention, coupled to a sensing mechanism for color analysis, said bag having a preferred forced air path to move breath across the sensing mechanism.

FIG. 4 is a side view of an alternate embodiment of a breath collection bag of the present invention, coupled to a sensing mechanism for color analysis, said bag having a preferred forced air path to move breath across the sensing mechanism.

### DESCRIPTION OF THE PREFERRED EMBODIMENT

Although the disclosure hereof is detailed and exact to enable those skilled in the art to practice the invention, the physical embodiments herein disclosed merely exemplify the invention which may be embodied in other specific structures. While the preferred embodiment has been described, the details may be changed without departing from the invention, which is defined by the claims.

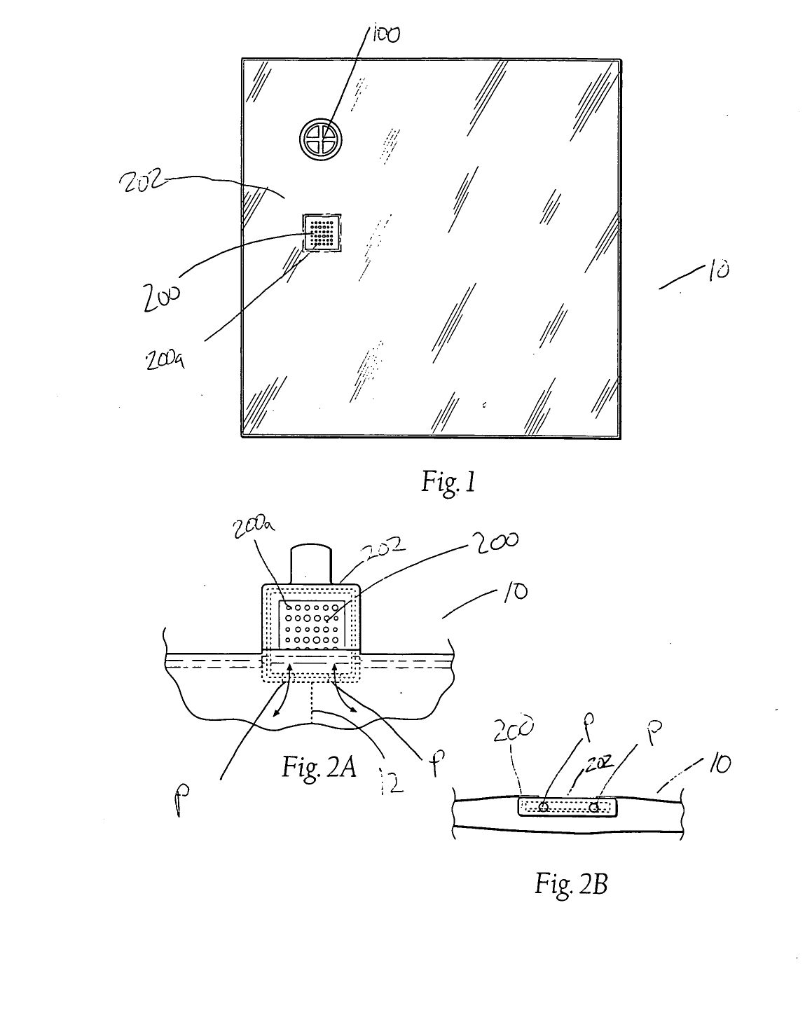

Referring now to FIG. 1, a side view of a breath collection bag 10 of the present invention, coupled to a sensing mechanism for color analysis is shown. A breath intake structure 100 is provided for a patient to breathe in, so that the breath can be exposed to the sensing mechanism 200. The sensing mechanism 200 should be at least partially exposed to the breath of the patient, so that the breath can be analyzed.

It is preferable, though not required, that the bag 10 is of two ply construction, the two plies sealed together. Of course, more or less plies may be used in the bag construction. The bag structure 10 is preferably expandable, and constructed of a supple, inert, and airtight material. One such supple and airtight material that performs suitably is thin polyester film, but foil laminate and a variety of other materials could also perform suitably. It should be easily recognizable to one skilled in the art that any number of materials other than foil laminate can be used for the bag structure 10. Other examples of materials that could be used for constructing the present invention include for illustration, but not by way of limitation: Tedlar®, Saranex®, Saran®, and Teflon®. Those skilled in the art will appreciate that the material can vary widely based on the characteristics of the gases desired to be sampled. The materials chosen should be inert and exhibit relative impermeability to the gases desired to sample, and any materials chosen that are relatively permeable to the gases desired to sample would not be preferred.

A preferred sensing mechanism 200 is an array-based vapor-sensing device, available from ChemSensing of Northbrook, Ill. This sensing mechanism is used to detect and differentiate between chemically diverse analytes, based on cross-responsive sensor elements to mimic the mammalian olfactory system by producing composite responses unique to each odorant, such as color response, as described in Rakow, N. A.; Suslick, K. S. “A Colorimetric Sensor Array for Odour Visualization” Nature, 2000, 406, 710-714; Suslick, K. S.; Rakow, N. A. “Colorimetric Artificial Nose Having an Array of Dyes and Method for Artificial Olfaction”, incorporated by reference. An array of dot shaped sensor elements 200a can detect a variety of substances within the breath of a human. There is described an optical chemical sensing method that utilizes the color change induced in an array of metalloporphyrin dyes upon ligand binding while minimizing the need for extensive signal transduction hardware. The chemoselective response of a library of immobilized vapor-sensing metalloporphyrin dyes permits the visual identification of a wide range of ligating (alcohols, amines, ethers, phosphines, phosphites, thioethers and thiols) and even weakly ligating (arenes, halocarbons and ketones) vapors.

One preferred sensing mechanism 200 comprises a device for detecting an analyte comprising an analyte-specific compound that binds to the analyte and produces a detectable compound in combination with a given substrate, said detectable compound producing a response when exposed to at least two dyes, the response being stronger and more distinct than a response of the analyte when exposed to the at least two dyes, such as disclosed in U.S. Patent Application 20040157281 to Hulkower, incorporated herein by reference.

For example, one or more porphyrin dyes in its metalated form (e.g., metalloporphyrin) or non-metalated form (e.g., free-base porphyrin) can be used to detect the enzymatically generated volatile compounds and the porphyrin dye can be used alone or as a component in an array with other chemical dyes, such as Bronsted acid-base dyes, Lewis acid-base dyes, zwitterionic solvatochromic dyes, and other chemoresponsive dyes. Those skilled in the art will recognize that any suitable method may be used to detect and quantitate a dye color change corresponding to the presence of the enzymatically generated volatile compound, such as the unassisted eye, spectrophotometry and fluorescence detection or other readers or sensors (not shown).

A human breath, after exposure to the sensing mechanism 200, will provide a change in color from a baseline color pattern, providing a unique color fingerprint at perceived and targeted analyte concentrations. The sensing mechanism 200 comprises a housing 202, preferably with a clear cover to allow easy perception of color change. The housing 202 holds the array 200a, and ports P (see FIG. 2a) allow air into the housing 202 for exposure to the array 200a.

The embodiment described in FIG. 1 is referred to as an ambient bag 10/sensor 200 system because the air is allowed to be exposed to the sensor 200 without a mechanism for moving the breath more quickly across the sensor 200. Dynamic systems will be described later, where the breath is actively passed across the sensor 200 to improve the speed that the sensor 200 can have enough breath past it to provide an accurate reading.

The preferred sensing mechanism 200 is shown as a preferred embodiment, others may do. The benefit of the sensing mechanism 200 coupled with the bag 10 is that the breath need not be transferred from the bag 10 to the testing medium—which could decrease test accuracy by introduction to contaminants, and also this assembly creates an easier test to administer.

FIG. 2A is a side view, with portions cut away, of an alternate embodiment of a dynamic system breath collection bag 10, coupled to a sensing mechanism 200 for color analysis is shown.

In this dynamic embodiment, the bag 10 is provided with a seam or partition 12 between two ports P. A user can then force air past the sensor array 200a by squeezing one side of the bag 10, alternating with the other side of the bag 10, in bellows like fashion. The air flow path would thus be from the left side of the bag 10 to the right, right to left and so on. In this embodiment, the housing 202 is sealed within a side seam of the bag 10, as opposed to the housing 202 within the limits of a face of the bag as shown in FIG. 1. A side seam or seam coupling is referred to herein as a sensing mechanism that is coupled between two seams of the bag 10.

The housing 202 can be sealed to the bag by such techniques as heat or adhesive.

FIG. 2B is a cross sectional view of a breath collection bag of the present invention shown in FIG. 1. In this embodiment, the housing 202 of the sensing mechanism 200 is coupled with the bag 10 in what is called face coupling or a face. Face coupling is referred to herein as a sensing mechanism that is coupled with one or more faces of the bag 10.

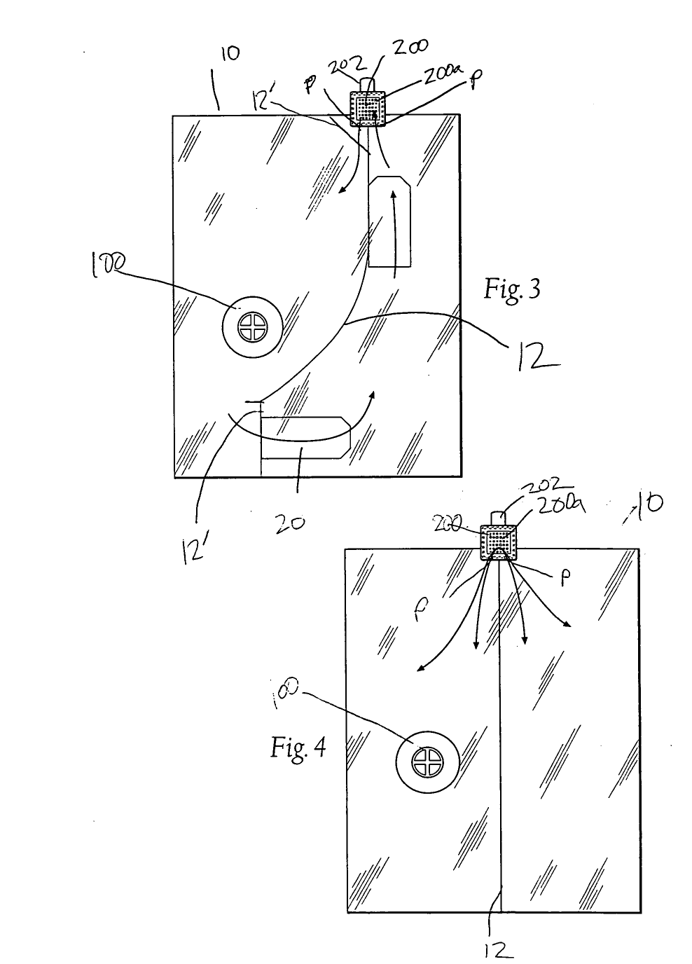

FIG. 3 is a side view of a dynamic seam coupling breath collection bag 10 of the present invention. In this embodiment, the air intake structure 100 is provided on one side of a partition 12. A preferred forced air path is provided by the partition 12, which divides right and left sides of the bag 10. A one way valve 20 to move breath across the sensing mechanism 200. A circular flow path is created in this embodiment. The partition 12 could be provided with zones of weakness 12′ across an inlet of the valve 20, and one of the ports P such that a single breath would be captured on one portion of the bag 10. By squeezing the bag 10, the zones of weakness 12′ would be ruptured, allowing manipulation of the bag 10 and air could be circulated across the circular flow path described.

FIG. 4 is a side view of an alternate embodiment of a seam sealed dynamic breath collection bag 10 of the present invention. In this embodiment, a right/left alternating flow path is created about the two sides of partition 12.

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention, which is defined by the claims.

I claim:

1. An air sampling apparatus comprising:
a bag for containing air;
a sensing mechanism coupled to said bag, said sensing mechanism having a housing coupled to said bag.

2. An air sampling apparatus according to claim 1, said housing and said bag coupled by a side seam.

3. An air sampling apparatus according to claim 2, said housing having at least two ports, said bag having a partition between said ports.

4. An air sampling apparatus according to claim 2, said housing having at least two ports, said bag having a partition between said ports; said partition having at least one zone of weakness.

5. An air sampling apparatus according to claim 2, said housing having at least two ports, said bag having a partition between said ports, said partition defining two interior portions of said bag, said interior portions coupled by a valve.

6. An air sampling apparatus according to claim 1, said housing and said bag coupled by a face coupling.

7. An air sampling apparatus according to claim 1, said apparatus comprising an ambient system.

8. An air sampling apparatus according to claim 1, said apparatus comprising a dynamic system.

9. An air sampling apparatus according to claim 1, wherein said sensing mechanism comprises a device for detecting an analyte comprising an analyte-specific compound that binds to the analyte and produces a detectable compound in combination with a given substrate, said detectable compound producing a response when exposed to at least two dyes, the response being stronger and more distinct than a response of the analyte when exposed to the at least two dyes.

10. A method of sampling a breath, said method comprising the steps of:
providing a bag with a breath intake structure and a sensing mechanism;
breathing into said bag;
allowing said breath to contact said sensing mechanism;
analyzing a response of said sensing mechanism to said breath.
